(19) United States
(12) Patent Application Publication
(10) Pub. No.: US 2025/0281034 A1
(43) Pub. Date: Sep. 11, 2025

(54) MEDICAL VISUALIZATION AND MONITORING DEVICES AND METHODS

(71) Applicants: GyneCheck Ltd., Mikhmoret (IL); Sheba Impact Ltd., Ramat-Gan (IL)

(72) Inventors: Einat Hila TZUR, Michmoret (IL); Shlomo HOLLANDER, Zichron-Yaakov (IL); Shlomi COHEN, Michmoret (IL)

(73) Assignees: GyneCheck Ltd., Mikhmoret (IL); Sheba Impact Ltd., Ramat-Gan (IL)

(21) Appl. No.: 18/862,540

(22) PCT Filed: May 3, 2023
(86) PCT No.: PCT/IL2023/050454

§ 371 Date: Nov. 3, 2024

Related U.S. Application Data
(60) Provisional application No. 63/337,648, filed on May 3, 2022.

(51) Int. Cl. A61B 1/05; A61B 1/00; A61B 1/015; A61B 1/018; A61B 1/045; A61B 1/06; A61B 1/303; A61B 1/32; A61B 10/04

(52) U.S. Cl. CPC A61B 1/05; A61B 1/00006; A61B 1/00016; A61B 1/0004; A61B 1/00082; A61B 1/00097; A61B 1/00119; A61B 1/00128; A61B 1/015; A61B 1/018; A61B 1/045; A61B 1/0676; A61B 1/303; A61B 1/32; A61B 10/04; A61B 2560/0462; A61B 2562/0219; A61B 2562/0247; A61B 2562/0271

(57) ABSTRACT

A medical diagnostic device may include: an annular body having a proximal end, a distal end, a central longitudinal axis and a hollow interior, and wherein the distal end is configured to be inserted into a bodily orifice of a subject; a base connected to the proximal end of the annular body adapted to be held by hand of the subject and having a hollow interior; a first imaging device insertable at the distal end of the annular body, wherein the first imaging device is configured to obtain one or more images of an internal environment of the bodily orifice of the subject; and a second imaging device insertable into the hollow interior of the base, wherein the second imaging device is configured to obtain one or more images of an external environment of the bodily orifice of the subject.

### FIELD OF THE INVENTION

The present invention relates to the field of medical visualization, and more particularly, to a medical diagnostic device for use in examining the internal and external environment of bodily orifices. In some embodiments, the device may be configured for vulvar-perineal-vaginal and cervical (VPVC) visualization and imaging. In some embodiments, the device may be adapted for use with other bodily orifices.

### BACKGROUND OF THE INVENTION

A periodic gynecological check-up is an important aspect of medical care for women, and for many women may be critical. The vaginal examination is often a part of routine periodic gynecological consultation. However, performance of the examination as a screening for gynecological diseases such as cancers and infections may be poor when performed in the asymptomatic patient. Cervical cancer screening may be appropriate every three years from the age of 24. However, the intrusive nature of vaginal examination can be experienced as traumatic or offensive by patients, and may discourage its use, for example, its use may become more targeted. Fear of the examination may also result in avoidance of care. Gynecological consultation is a privileged setting for screening for sexual risk taking or domestic violence, but also for exploring overall sexual health and promoting women's health.

The need for appropriate visualization, monitoring and diagnostic tools for gynecological examination is critical. For example, Invasive Cervical Cancer (ICC) is the second most common female cancer in low and middle-income countries (LMICs) and the seventh most common in high-income countries. Annually, over 500,000 women are diagnosed, causing over 270,000 deaths recorded with more than 75% of cases occurring in Africa and India. The World Health Organization (WHO) estimates that currently 88% of worldwide ICC mortalities occur in LMICs, and this rate is expected to increase to 98% by 2030, furthering the disparities as the total number of annual worldwide mortalities increases to nearly 400,000. Though early diagnosis and treatment of cervical pre-cancers have been shown to significantly increase survival rates, diagnostic tools are not widely available in LMICs.

Currently, the standard-of-care screening method in most LMICs is visual inspection with acetic acid (VIA). This technique involves the use of a speculum to expand the vaginal canal to enable a clear field-of-view of the cervix, for visualization with a colposcope, camera or directly by the health provider (naked eye). Speculums are needed mainly because of the need to expand the entire vaginal canal.

The speculum has been identified as a significant factor in the resistance of women to undergo cervical cancer screening, largely due to anxiety, fear, discomfort, pain, embarrassment, and/or vulnerability during the procedure. In the U.S., even though there is greater access to health care, compliance rates to cervical cancer screening vary, and embarrassment and fear of pain during examination have been reported as potential barriers to screening.

The speculum has been in existence in various shapes and forms since the tenth century and has evolved with hundreds of modifications in attempts to enhance exposure. The first rudimentary prototype of the modern speculum was developed out of a bent spoon. The semblance to the standard bivalve speculum was put forward by the manufacturer Charriere who introduced the bivalve, tri-blade and four-blade speculum. This inspired the duck bill designs of the familiar Cusco speculum in 1870 and the Graves speculum in 1878. These are cold, hard, metal devices with two bills each that expand the entire vaginal canal. Since the introduction of duck billed speculums, there have been few improvements to make them more comfortable and acceptable for women. Slight changes in design have involved introducing a variety of sizes and making the speculum out of plastic. Current speculums are designed for an external user, which makes it difficult for self-insertion by women, and therefore also difficult to re-adjust when there is discomfort. Furthermore, in cases where women have tilted uteri or lax vaginal walls due to having a larger body size or a high parity, increased manipulation or use of an extra device, such as a side wall retractor, is needed to obtain a clear view of the cervix. This may further add to discomfort and pain during vaginal examinations.

U.S. Pat. No. 10,694,954 teaches a system for imaging and diagnosing target internal

tissues including an image acquisition system mechanically secured to a speculum. This solves certain diagnostic imaging issues but does not address the drawbacks of the speculum.

U.S. Patent Application Publication No. 2019/0150725 describes speculum-free imaging systems for colposcopes and mammoscopes, but does not address insertion by the user or visualization of an exterior of a body cavity.

### SUMMARY OF THE INVENTION

Recently, the global COVID-19 pandemic has highlighted the need for a remote physician-mediated gynecological pelvic examination, obtainable by the patient at their convenience and in an appropriate location without the need for scheduling an office visit, thereby avoiding all the problems associated with an in-person office visit, as well as time spent traveling to the gynecologist's office, and waiting, for a simple annual or urgent check-up. Thus, in one aspect, the invention may be embodied as a self-examination kit with no need for a speculum, providing immediate answers to the majority of both urgent and chronic common gynecological problems.

In one embodiment, a device according to embodiments of the invention may be used by the patient for self-examination of both urgent and chronic issues such as: vulvovaginal infection; sexually transmitted disease (STD); non-periodic and periodic heavy and irregular bleeding; vulvar pain; vulvar itch; forensic data collection such as in instances of sexual harassment and rape; carly and late pregnancy bleedings; detection of ovulation by, for example, analyzing cervical mucus and Basal Body Temperature (BBT); and suspected amniotic fluid loss during pregnancy (PPROM-pre mature rupture of membranes). The device can be used during monitoring of labor and delivery to assess cervical dilation and effacement as well as progression of the fetal head and fetal heartbeat. In embodiments, the device may incorporate an ultrasound probe disposed at a distal end thereof for urgent diagnoses such pregnancy vitality or location (in the case of ectopic pregnancy for example).

In another embodiment, a device according to of the invention may be used by the patient for self-examination, e.g., of chronic pathologies, and/or in the course of annual check-ups, e.g., including: annual visual check-up for anatomical aberrations like vaginal wall, urinary bladder, rectum and uterine prolapses (using pressure and size sensors mounted on the device as described below); periodic screening for HPV infections (all three routes: vulvar, vaginal and cervical); early detection of vulvar lesions such as lichen sclerosis, VAIN, Vulvar and Vaginal pre-malignant and malignant changes; chronic pruritus: vulvovaginal yeast infection and atrophic vaginitis; and diagnosis of chronic conditions such as endometriosis and Chronic Pelvic Pain (CPP). In embodiments, sensors may be added to the apparatus for inspection of pressure, pH, impedance, and/or performing electromyography (EMG) (for detection of nerve fibers pain and mechanic function). These elements may allow for a robust diagnostic tool providing visualization and information in real time that the patient may use in her own space.

In embodiments, the device may be provided with a direct connection to both patient and physician via smartphone, PC, tablet, or other computing device using artificial intelligence (AI) to facilitate self-examination or gynecologist-mediated diagnosis. Thus, a patient may decide when to rely on Al analysis and off-the-counter solutions for common acute clinical conditions such as candidiasis or other vaginal infections, when to schedule a remote consultation with their regular gynecologist or an urgent consultation with a specialist on call. Diagnostic information obtained from sensors on the device, or data collected via swabs can be transmitted to and from the medical diagnostic device and to and from remote computing devices. Likewise, mechanical controls in the device may be operable by the subject hand or may be operable remotely with a remote computing device.

In some embodiments, a medical diagnostic device according to the invention comprises an annular body having a proximal end, a distal end and a hollow interior, wherein the distal end is configured to be inserted into a bodily orifice of a subject; a base connected to the proximal end of the annular body adapted to be held by hand of the subject and having a hollow interior; a first imaging device insertable at the distal end of the annular body, wherein the first imaging device is configured to obtain one or more images of an internal environment of the bodily orifice of the subject; and a second imaging device insertable into the hollow interior of the base, wherein the second imaging device is configured to obtain one or more images of an external environment of the bodily orifice of the subject.

In embodiments, controls on the base may be adapted to be manipulated by hand to rotate the first imaging device vertically and laterally and about a longitudinal axis of the annular body, and in other embodiments, movement of the first imaging device may be controlled remotely. The first imaging device may be adapted to be rotatable in at least three planes of motion, and in embodiments to provide a field of view ranging from 5 mm to 30 cm.

In embodiments, the device may further comprise: a first inflatable member connected to the annular body; a first conduit connected to the first inflatable member and adapted to inflate the first inflatable member; wherein the first inflatable member is an annular member surrounding the annular body.

In embodiments, the device may further comprise: controls, operable from the base, by

hand or remotely, configured to inflate and deflate the first inflatable member at different speeds.

In embodiments, the device may further comprise: a second inflatable member, wherein the second inflatable member is connected to the first inflatable member; a second conduit connected to the second inflatable member adapted to inflate the second inflatable member to a diameter greater than a diameter of the first inflatable member. In embodiments, the device may further comprise: controls, operable from the base or remotely, configured to inflate and deflate the first inflatable member according to the size and anatomy of the bodily orifice.

In embodiments, the device may further comprise: an inflatable anchoring member operable to anchor the annular body in the bodily orifice.

In embodiments, the device may further comprise: legs or wings housed in the annular body configured to anchor the annular body.

In embodiments, the second inflatable member is an annular member surrounding a portion of the first inflatable member at the distal end of the annular body.

In embodiments, the device may further comprise: a resilient member fitting tightly within the interior of the annular body, the resilient member may be fully accommodated within the interior of the annular body and configured to partly protrude from the distal end of the annular body, the resilient member comprising: a support; and one or more pairs of opposed longitudinal elements cach connected at one end to the support; wherein the resilient member is preloaded such that when the longitudinal elements protrude from the distal end of the annular body, at least one of the longitudinal elements recoils away from the central longitudinal axis of the annular body such that a distance formed between free ends of the longitudinal elements is greater than an outer diameter of the distal end of the annular body.

In embodiments, the first imaging device may be connected to a motor in the base configured to change an angle of the first imaging device with respect to the bodily orifice of the subject.

In embodiments, the device may further comprise: a swabbing member fitting within the interior of the annular body, being fully accommodated within the interior of the annular body and configured to protrude from and withdraw into the distal end of the annular body, the swabbing member comprising: a support; and a swab connected to the support. In embodiments, the swabbing member is operable from the base to push the support of the swabbing member to cause the swab of the swabbing member to protrude from the distal end of the annular body. The swab may be used for cleaning the vaginal cervical secretions to permit optimal visualization of vaginal walls and cervix.

In embodiments, the device may further comprise: a longitudinal channel within the annular body and extending between the proximal end and the distal end of the annular body, wherein the longitudinal channel is configured to receive a tool or a tool within an envelope.

In embodiments, the device comprises: a longitudinal channel surface connected to an inner longitudinal surface of the annular body such that a hollow channel interior is formed between the longitudinal channel surface and the inner longitudinal surface of annular body; wherein the channel interior is configured to receive the tool or the tool within the envelope.

In embodiments, the device comprises: a longitudinal channel surface connected to an outer longitudinal surface of the annular body such that a hollow channel interior is formed between the longitudinal channel surface and the outer longitudinal surface of annular body; and the channel interior is configured to receive the tool or the tool within the envelope. The longitudinal channel may be formed in a wall of the annular body.

In embodiments, the device comprises: one or more sensors disposed on an outside surface of the annular body, the one or more sensors configured to measure a set of parameters of the internal environment of the bodily orifice of the subject comprising at least one of impedance, pH and pressure. In embodiments, an EMG electrode may be disposed on the annular body.

In embodiments, the annular body is comprised of two or more interconnected annular segments, cach having a proximal end, a distal end, a hollow interior and a central longitudinal axis; wherein in each pair of a first adjacent annular segment and a second adjacent annular segment, the second adjacent annular segment is rotatably connected at its proximal end to the distal end of the first adjacent annular segment of the respective pair such that the central longitudinal axis of the second adjacent annular segment is displaceable with respect to the central longitudinal axis of the first adjacent annular segment of the respective pair; and wherein first imaging device may be insertable into interiors of annular segments.

The device may further comprise a guiding wire to guide the movement of the first imaging device within the interiors of the two or more annular segments.

In embodiments, the device comprises: a DC power supply comprising at least one of a battery, electric bus power supply interface and induction power supply interface; a pump connected to the first conduit and to the second conduit; and sensors configured to control operation of the pump; wherein the DC power supply, the pump and the sensors are insertable into the hollow interior of the basc.

In embodiments, the pump is configured to: regulate the flow of pressurized gas through the first conduit to inflate and deflate the first inflatable member; and regulate the flow of pressurized gas through the second conduit to inflate and deflate the second inflatable member.

In embodiments, the device comprises: a fiber-optic cable insertable into the hollow interior of the annular body, wherein the fiber-optic cable comprises a proximal end and a distal end. The proximal end of the fiber-optic cable may terminate within the hollow interior of the base and the distal end of the fiber-optic cable may terminate at the distal end of the annular body, and wherein the distal end of the fiber-optic cable is adapted to connect to at least one of an ultrasound probe or a third imaging device.

In embodiments, the device may comprise a disposable or reusable clastic transparent cover on an end of the longitudinal channel at the distal end of the annular body.

In embodiments, the first imaging device may be adapted to: scan and obtain images of the internal environment of the bodily orifice of the subject in at least three planes of motion according to a predetermined pattern.

In embodiments, the first imaging device may be further adapted to obtain images of a predefined set of anatomical features, which may include a panoramic pelvic view, a vulvar-vestibular panoramic view, a vagina panoramic view, a posterior fornix view, and an anterior fornix view.

In embodiments, the device may comprise a processor; and a communication unit comprising at least one of a wireless and a non-wireless communication interface; wherein the processor and communication unit are disposed in the base.

In embodiments, the processor may be configured to: control a process of the first imaging device obtaining images of the predefined set of anatomical features; generate status data relating to the process of the first imaging device obtaining images of the predefined set of anatomical features; generate and update a personalized profile of the subject from the images; and communicate the status data and the personalized profile to a remote computing device using the communication unit.

In embodiments, the status data may comprise at least an indication of successfully obtaining images of one anatomical feature from the predefined set of anatomical features and an indication of successfully obtaining images of all anatomical features from the predefined set of anatomical features.

In embodiments, the device may comprise an accelerometer and/or a gyroscope to measure orientation and location of the first imaging device within the bodily orifice of the subject.

In some embodiments, a medical diagnostic device may comprise: an annular body having a proximal end and a distal end, wherein the distal end is configured to be inserted into a bodily orifice of a subject; an expandable member connected to the annular body, wherein the expandable member is configured to expand at least a portion of the bodily orifice of the subject; and an imaging device insertable at the distal end of the annular body, wherein the imaging device is configured to obtain one or more images of an internal environment of the bodily orifice of the subject.

In some embodiments, a method for medical observations of a bodily orifice of a subject using a medical diagnostic device may comprise: inserting into a bodily orifice of a subject, a distal end of an annular body, wherein the annular body comprises the distal end and a proximal end; expanding at least a portion of the bodily orifice of the subject using an expandable member connected to the annular body; and obtaining one or more images of an internal environment of the bodily orifice of the subject using an imaging device insertable at the distal end of the annular body.

These, additional, and/or other aspects and/or advantages of the present invention are set forth in the detailed description which follows; possibly inferable from the detailed description; and/or learnable by practice of the present invention.

### BRIEF DESCRIPTION OF THE DRAWINGS

For a better understanding of embodiments of the invention and to show how the same can be carried into effect, reference will now be made, purely by way of example, to the accompanying drawings in which like numerals designate corresponding elements or sections throughout.

In the accompanying drawings:

FIG. 1 shows a block diagram of an example computing device, according to some embodiments of the invention;

FIGS. 2A-2C are schematic illustrations of a medical diagnostic device, according to some embodiments of the invention;

FIG. 3 is an exploded view of a device according to embodiments of the invention showing a camera tilt-rotation mechanism;

FIGS. 4A-4D are schematic illustrations of a medical diagnostic device including first and second inflatable members, according to some embodiments of the invention;

FIGS. 5A-5E are schematic illustrations of a medical diagnostic device including a resilient member, according to some embodiments of the invention;

FIGS. 6A-6D are schematic illustrations of a medical diagnostic device including a swabbing member, according to some embodiments of the invention;

FIGS. 7A-7F are schematic illustrations of a medical diagnostic device including a longitudinal channel, according to some embodiments of the invention;

FIG. 8 is a schematic illustration of a medical diagnostic device including one or more sensors, according to some embodiments of the invention;

FIGS. 9A and 9B are schematic illustrations of a medical diagnostic device, wherein the annular body includes two or more interconnected annular bodies, according to some embodiments of the invention;

FIG. 10 is a schematic illustration of an inflatable member and associated pump according to some embodiments of the invention; and

FIG. 11 shows a flowchart representing a method for screening or imaging of an orifice of a subject according to embodiments of the present invention.

It will be appreciated that, for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following description, various aspects of the present invention are described. For purposes of explanation, specific configurations and details are set forth in order to provide a thorough understanding of the present invention. However, it will also be apparent to one skilled in the art that the present invention can be practiced without the specific details presented herein. Furthermore, well known features can have been omitted or simplified in order not to obscure the present invention. With specific reference to the drawings, it is stressed that the particulars shown are by way of example and for purposes of illustrative discussion of the present invention only and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention can be embodied in practice.

Before at least one embodiment of the invention is explained in detail, it is to be understood that the invention is not limited in its application to the details of construction and the arrangement of the components set forth in the following description or illustrated in the drawings. The invention is applicable to other embodiments that can be practiced or carried out in various ways as well as to combinations of the disclosed embodiments. Also, it is to be understood that the phraseology and terminology employed herein is for the purpose of description and should not be regarded as limiting.

Although embodiments of the invention are not limited in this regard, discussions utilizing terms such as, for example, “processing”, “computing”, “calculating”, “determining”, “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

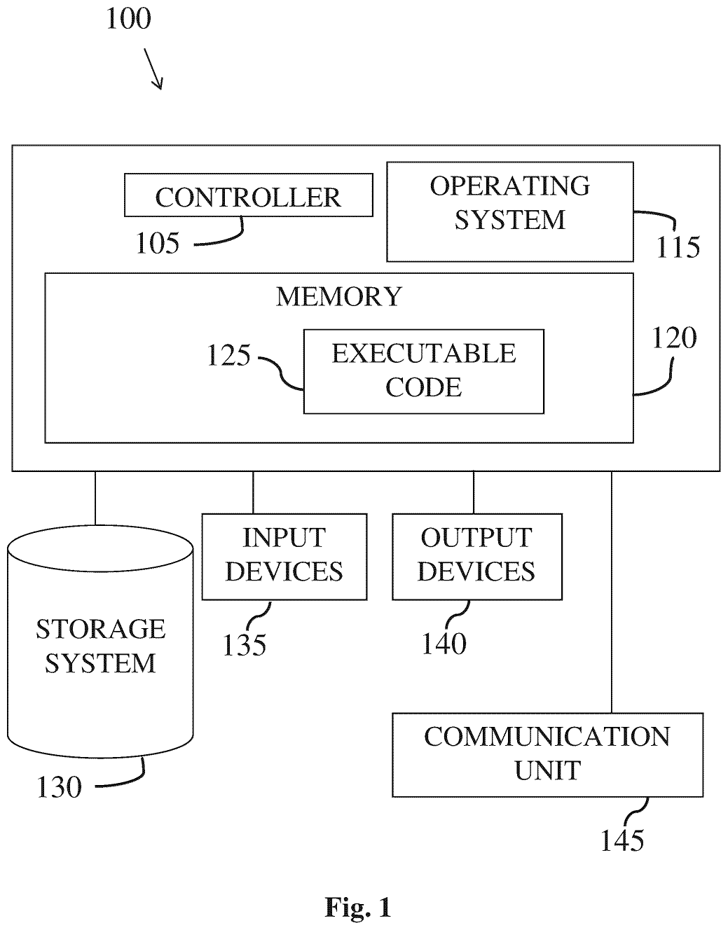

Reference is made to FIG. 1, which is a schematic block diagram of an example computing device, according to some embodiments of the invention. Computing device 100 may include a controller or processor 105 (e.g., a central processing unit processor (CPU), a chip or any suitable computing or computational device), an operating system 115, memory 120, executable code 125, storage 130, input devices 135 (e.g. a keyboard or touchscreen), and output devices 140 (e.g., a display), a communication unit 145 (e.g., a cellular transmitter or modem, a Wi-Fi communication unit, or the like) for communicating with remote devices via a communication network, such as, for example, the Internet. Controller 105 may be configured to execute program code to perform operations described herein. Systems or devices described herein may include one or more computing device(s) 100. For example, systems/devices 200, 300, 400, 500, 600, and 700 may include computing device 100 or components thereof.

Operating system 115 may be or may include any code segment (e.g., one similar to executable code 125 described herein) designed and/or configured to perform tasks involving coordinating, scheduling, arbitrating, supervising, controlling or otherwise managing operation of computing device 100, for example, scheduling execution of software programs or enabling software programs or other modules or units to communicate.

Memory 120 may be or may include, for example, a Random Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 120 may be or may include a plurality of, possibly different, memory units. Memory 120 may be a computer or processor non-transitory readable medium, or a computer non-transitory storage medium, e.g., a RAM.

Executable code 125 may be any executable code, e.g., an application, a program, a process, task or script. Executable code 125 may be executed by controller 105 possibly under control of operating system 115. For example, executable code 125 may be a software application that performs methods as further described herein. Although, for the sake of clarity, a single item of executable code 125 is shown in FIG. 1, a system according to embodiments of the invention may include a plurality of executable code segments similar to executable code 125 that may be stored into memory 120 and cause controller 105 to carry out methods described herein.

Storage 130 may be or may include, for example, a hard disk drive, a universal serial bus (USB) device or other suitable removable and/or fixed storage unit. In some embodiments, some of the components shown in FIG. 1 may be omitted. For example, memory 120 may be a non-volatile memory having the storage capacity of storage 130. Accordingly, although shown as a separate component, storage 130 may be embedded or included in memory 120.

Input devices 135 may be or may include a keyboard, a touch screen or pad, one or more sensors or any other or additional suitable input device. Any suitable number of input devices 135 may be operatively connected to computing device 100. Output devices 140 may include one or more displays or monitors and/or any other suitable output devices. Any suitable number of output devices 140 may be operatively connected to computing device 100. Any applicable input/output (I/O) devices may be connected to computing device 100 as shown by blocks 135 and 140. For example, a wired or wireless network interface card (NIC), a universal serial bus (USB) device or external hard drive may be included in input devices 135 and/or output devices 140.

Embodiments of the invention may include an article such as a computer or processor non-transitory readable medium, or a computer or processor non-transitory storage medium, such as for example a memory, a disk drive, or a USB flash memory, encoding, including or storing instructions, e.g., computer-executable instructions, which, when executed by a processor or controller, carry out methods disclosed herein. For example, an article may include a storage medium such as memory 120, computer-executable instructions such as executable code 125 and a controller such as controller 105. Such a non-transitory computer readable medium may be for example a memory, a disk drive, or a USB flash memory, encoding, including or storing instructions, e.g., computer-executable instructions, which when executed by a processor or controller, carry out methods disclosed herein. The storage medium may include, but is not limited to, any type of disk including, semiconductor devices such as read-only memories (ROMs) and/or random access memories (RAMs), flash memories, electrically erasable programmable read-only memories (EEPROMs) or any type of media suitable for storing electronic instructions, including programmable storage devices. For example, in some embodiments, memory 120 is a non-transitory machine-readable medium.

A system according to embodiments of the invention may include components such as, but not limited to, a plurality of central processing units (CPU) or any other suitable multi-purpose or specific processors or controllers (e.g., controllers similar to controller 105), a plurality of input units, a plurality of output units, a plurality of memory units, and a plurality of storage units. A system may additionally include other suitable hardware components and/or software components. In some embodiments, a system may include or may be, for example, a personal computer, a desktop computer, a laptop computer, a workstation, a server computer, a network device, or any other suitable computing device.

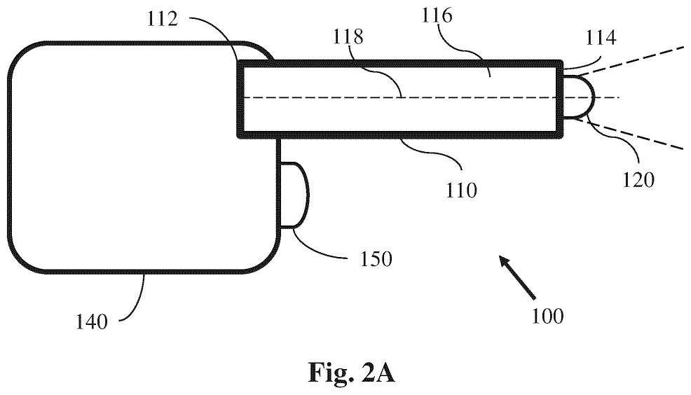

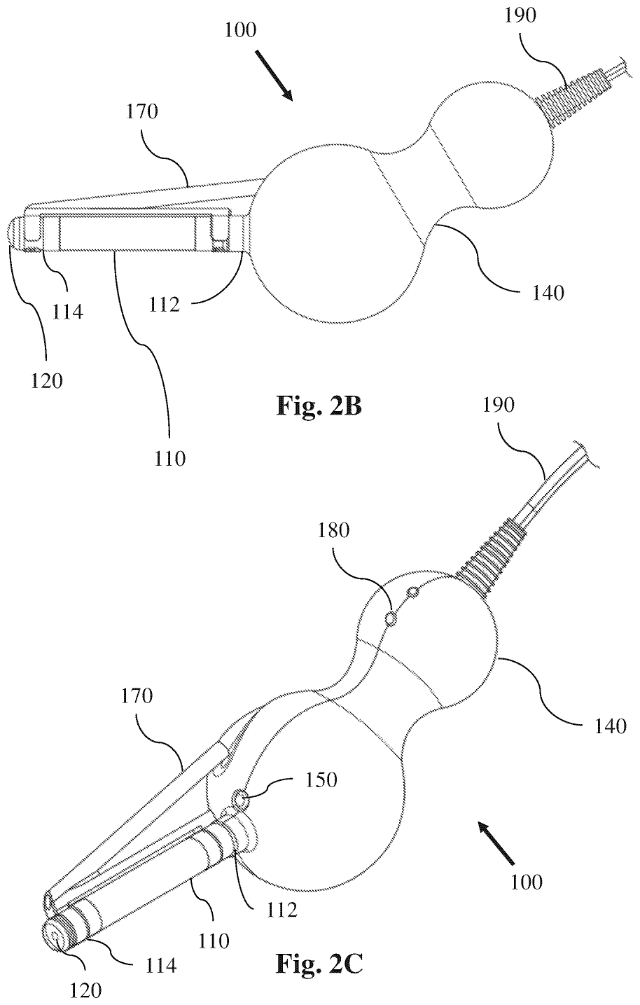

Reference is now made to FIGS. 2A-2C, which are schematic illustrations of a medical diagnostic device (100), such as a colposcope, according to some embodiments of the invention.

According to some embodiments of the invention, the medical diagnostic device may include an annular body (110), a base (140), a first imaging device (120) and/or a second imaging device (150). The annular body may be made of a biocompatible material, such as stainless steel, medical grade silicone, other medical grade polymers, medical grade ceramics and other materials known to those having skill in the art suitable for insertion into a body cavity. The annular body may be shaped and sized for insertion into the body cavity with which the device is used-requiring only a narrow channel to accommodate a first imaging device. The annular body may be rigid or flexible and/or may be made of movable segments to allow bending of the annular body in the body cavity.

The annular body may have a proximal end (112), a distal end (114), a hollow interior (116) and a central longitudinal axis (118). The distal end of the annular body may be configured to be inserted into a bodily orifice of a subject and the proximal end of the annular body may be connected to the base of the device, which may be adapted to be held by hand of the subject.

The annular body may have or may be connected to an expandable member, wherein the expandable member is configured to expand at least a portion of the bodily orifice of the subject when the annular member is inserted in the bodily orifice. Expanding and retracting of the expandable member may be configured to be controlled by controls (e.g., buttons or a touch interface) (180), which may be mounted on or operable from the base. Expansion of at least a portion of the bodily orifice may be preferable for capturing images of a bodily orifice of a subject.

The base may be shaped ergonomically to be gripped by the user, which may be useful, for example, when a subject performs self-examination. One or more processors and/or computing devices (such as those described in FIG. 1) may be disposed in the base interior and may be adapted to transmit data (e.g., images) obtained from the first and/or second imaging devices to a remote computing device and/or display. One or more processors or computing devices in the base may be adapted for controlling rotation of the first imaging device in three planes responsive to hand controls (180) on the base or remote controls. Electronic controls, a pump, and/or a power supply may be disposed in the base.

FIGS. 2B-2C provide an example of a shape of the base according to some embodiments

of the present invention, wherein FIG. 2B provides a side view and FIG. 2C provides a diagonal view. The base may have the shape of two substantially sphere-shaped portions, which may be connected by a continuous curved surface. The two substantially sphere-shaped parts may be of a different size. The shape of the base may be configured to be help comfortably by a human hand (e.g., the shape may be ergonomic).

The first imaging device (e.g., a charge coupled device (CCD) or a complementary metal oxide semiconductor (CMOS) device) may be disposed in the hollow interior of the annular body at the distal end of the annular body, wherein first imaging device is configured to obtain one or more images of an internal environment of the bodily orifice of the subject, e.g., images of vaginal walls, uterus, cervix, etc. Alternatively, or in addition to the first imaging device, the device may incorporate an ultrasound transducer in a “chip-on-tip” configuration. In FIG. 2A, the diagonal dashed lines emanating from the first imaging device may indicate the field of view of the imaging device.

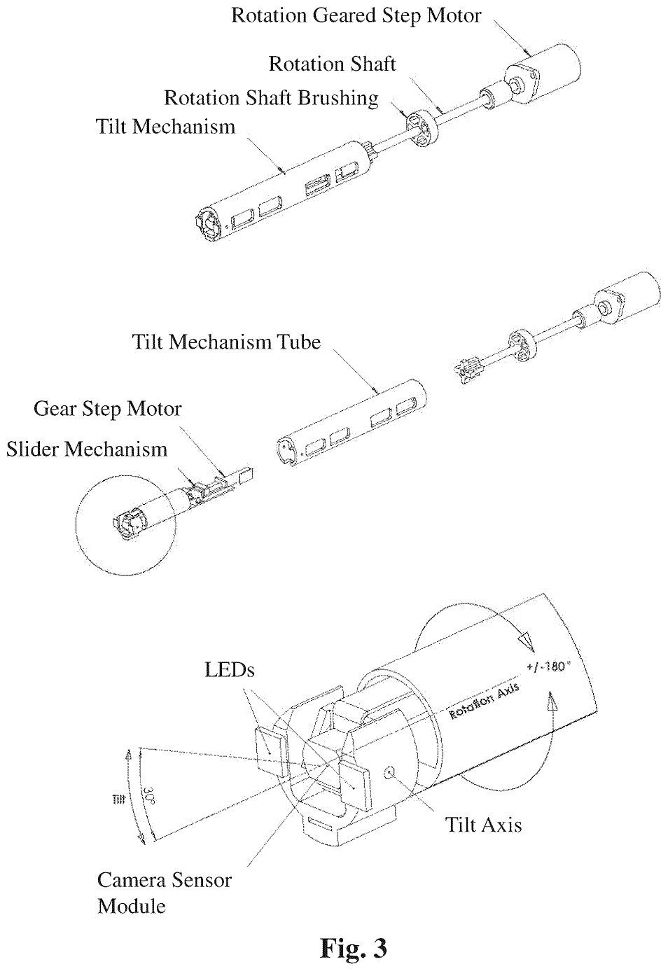

The first imaging device may be movable within the interior of the annular body. For example, the first imaging device may be movable in a longitudinal direction, tilted with respect to central longitudinal axis of annular body, rotated about the central longitudinal axis in a plane parallel to the axis and rotated about the central longitudinal axis in a plane perpendicular to the central longitudinal axis of annular body. Thus, controls for the first imaging device, operated by hand controls or remotely, may allow rotation about three axes: lateral, vertical and rotational about the central axis. (When referring to the first imaging device, for purposes of orientation, when the annular body is flexed, the longitudinal axis is the axis at the distal end of the annular body where the first imaging device is disposed.) The first imaging device may be as described with respect to FIG. 3.

The first imaging device may obtain a plurality of images of the internal environment of the bodily orifice of the subject (e.g., an intravaginal environment), wherein each of the plurality of images may include a different view of the internal environment. The image data may be processed remotely, for example using Artificial Intelligence (AI), to evaluate the clinical relevance of the data and/or as feedback to direct and control positioning of the first imaging device, using a smartphone, tablet, PC, or other computing device.

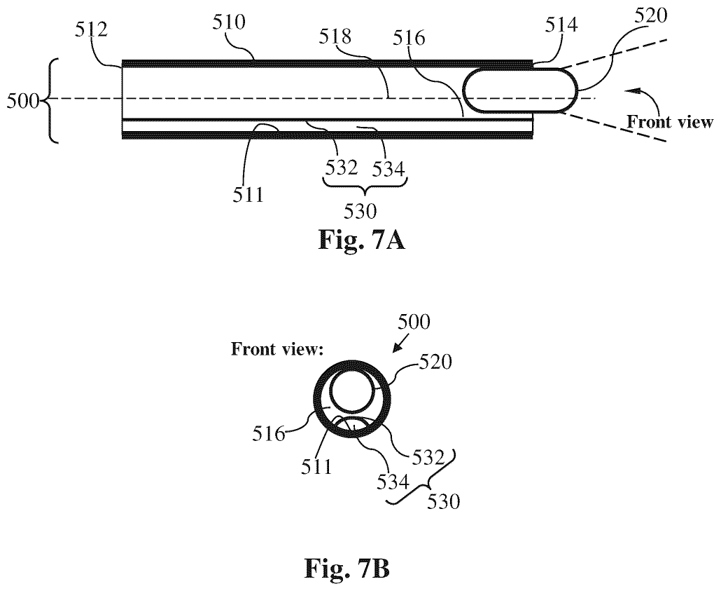

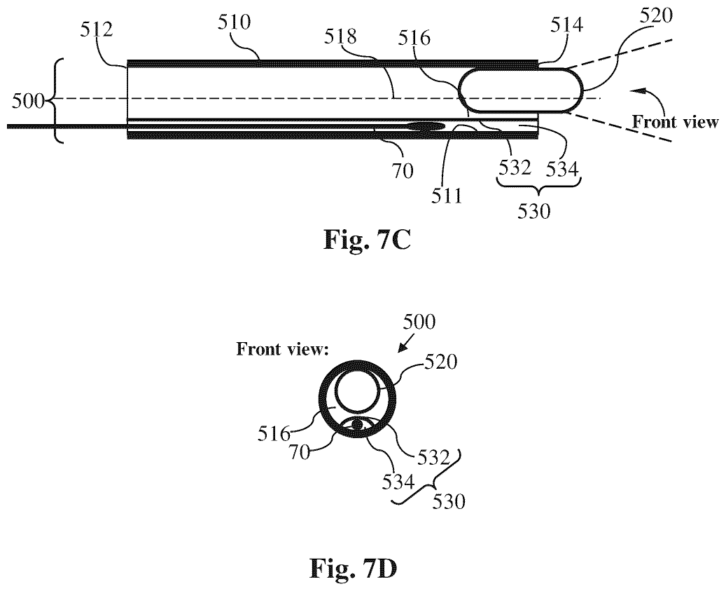

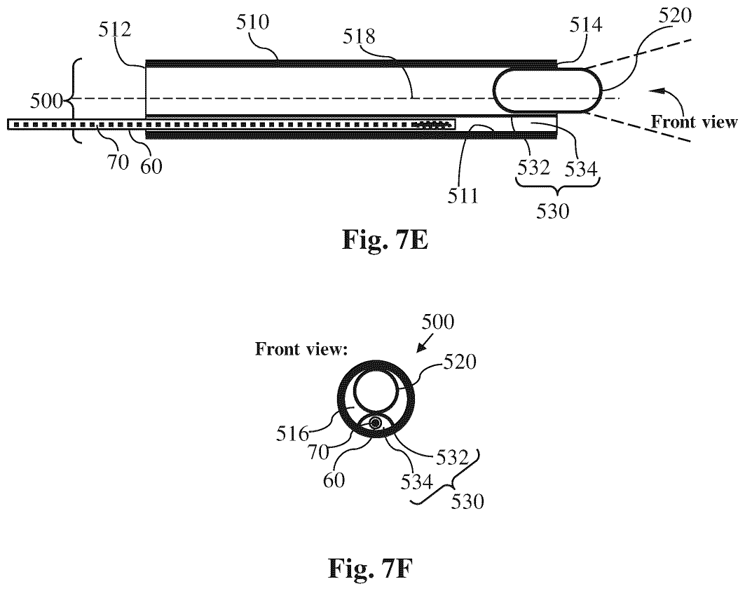

FIGS. 2D-2C further show an optional work channel (170), which may be configured to receive a tool and to enable insertion of the tool into a bodily orifice of a subject. The tool may include, for example, a test swab, biopsy forceps, etc. In some embodiments, the longitudinal channel may be configured to receive a tool within an envelope and to enable insertion of the tool and the envelope into the bodily orifice. The work channel may be as described in FIGS. 7A-7F. The work channel may be constructed of a flexible material, and may be configured to flex or deform when inserted into the bodily orifice.

FIGS. 2D-2C further depict an optional set of expandable member control buttons (180), configured to be mounted on the base, and configured to allow a user to expand and contract an expandable member of the apparatus. In alternative embodiments, the buttons may be disposed in another location, or a different control input may be used, e.g., a computer with input and output devices.

FIGS. 2D-2C further depict one or more optional cables (190) configured to carry data (e.g., image data) between the medical diagnostic device and a second device, e.g., a computer, and/or configured to carry power to the medical diagnostic device, e.g., in order to power components, such as a camera or air pump. In other embodiments, cables may not be required, for example, the medical diagnostic device may convey or receive data wirelessly and may be powered via internal batteries.

FIG. 3 depicts a camera tilt rotation mechanism in an exploded view, according to some embodiments of the invention. In this embodiment, a camera or camera sensor module may be operatively connected to a slider mechanism having at least one rail/tube guiding longitudinal movement within annular body. A camera assembly may be mounted in a tilt mechanism tube. Rotation of the camera view may be controlled by a rotation geared step motor, a rotation shaft and rotation shaft bushing operating within the tilt mechanism tube. Tilting of the camera view may be controlled by gyroscope-style bracket having a tilt axis perpendicular to the longitudinal axis of tilt mechanism tube about which the camera sensor may rotate. A transparent cover may be disposed at the distal end of the annular body to protect the probe and associated light source (e.g., one or more LEDs). In another embodiment a spherical camera may be used.

The second imaging device (e.g., a camera or probe) may be disposed in a hollow interior of the base and configured to obtain one or more images of an external environment of the bodily orifice of the subject. Data (e.g., image data) from the second imaging device may be processed by one or more processors in the base and transmitted to one or more remote processors in the same way as data obtained from the first imaging device or probe.

In some embodiments, the first imaging device and/or second imaging device may be connectable to a remote computing device. The first imaging device and/or second imaging device may transmit the images to remote computing device. Remote computing device may be, for example, a tablet, a smartphone, a personal computer, etc. In some embodiments, a remote computing device may present images to a user (e.g., the subject and/or a healthcare professional). In some embodiments, remote computing device may combine at least two images of at least two different views into a panoramic image including a panoramic view. In some embodiments, remote computing device may present the panoramic image to the user. In some embodiments, remote computing device may transmit the images and/or the panoramic image to a remote third party (e.g., a healthcare professional at a remote location) for clinical evaluation or other purpose.

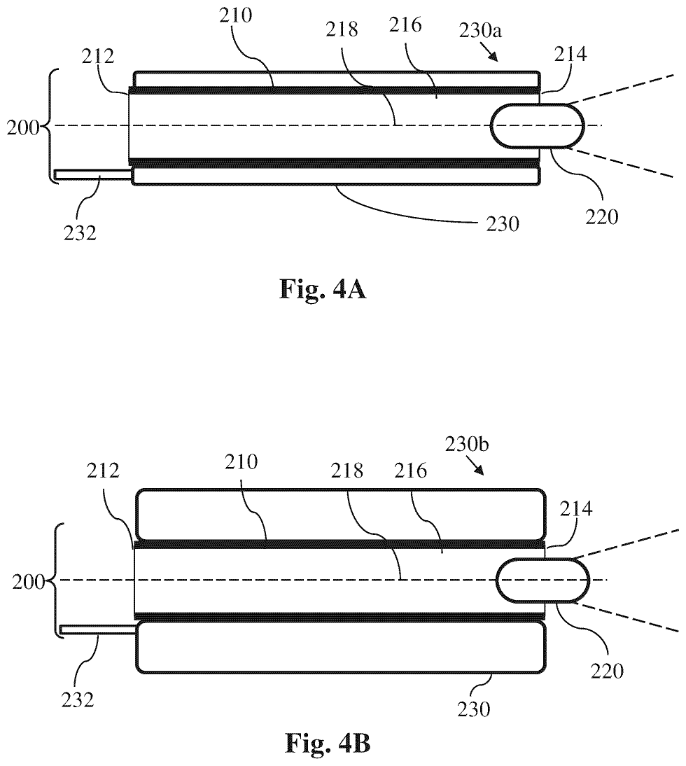

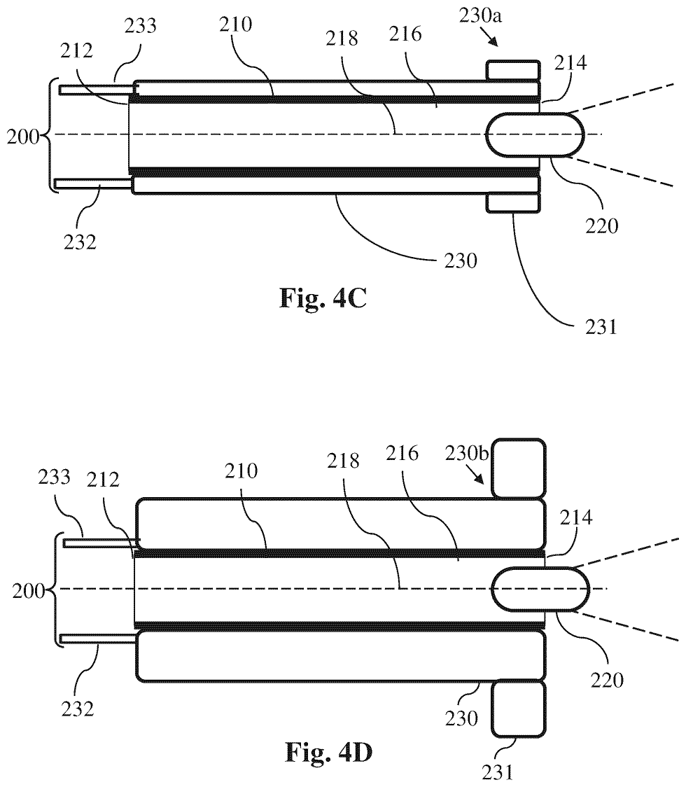

Reference is now made to FIGS. 4A-4D, which are schematic illustrations of a medical diagnostic device (200) including first (230) and/or second (231) inflatable members, according to some embodiments of the invention. In embodiments, the inflatable members may be embodiments of expandable members, operated to separate walls of a body cavity and expand a channel to facilitate image acquisition. In embodiments, secondary inflatable members may be used to provide improved access and visualization, for example, when the subject is obese or suffers from a prolapse.

The medical diagnostic device may include an annular body (210), a first imaging device (220), and may have a proximal end (212), a distal end (214), a hollow interior (216) and a central longitudinal axis (218). The distal end of the annular body may be configured to be inserted into a bodily orifice of a subject and the proximal end of the annular body may be connected to a base of the device, which may be adapted to be held by hand of the subject or user.

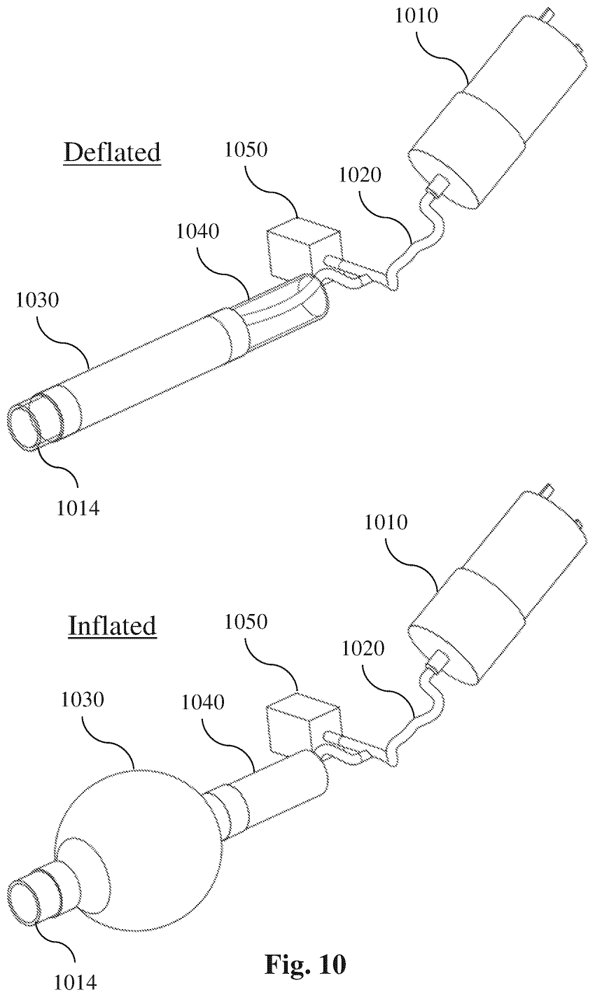

According to some embodiments of the invention, the medical diagnostic device may include a first inflatable member (230) connected to the annular body, and a first conduit (232), the first conduit connected to a pressure source (e.g., a pump) and the first inflatable member, and adapted to inflate the first inflatable member. In some embodiments, the first inflatable member is an annular member surrounding the annular body. In some embodiments, the medical diagnostic device may include controls configured to inflate and deflate the first inflatable member at different speeds, for example a first setting may allow for gradual inflation and deflation while a second setting permits immediate (“emergency”) deflation. In some embodiments, the medical diagnostic device may include controls, for example, operable from a base, by hand or remotely, configured to inflate and deflate the first inflatable member according to the size and anatomy of the bodily orifice. For example, FIG. 10 depicts an inflatable member surrounding at least a portion of the annular body, in a deflated and an inflated state, as well as the pump and release valve for inflating and deflating the device at different settings (which may be located within the base).

According to some embodiments of the invention, the medical diagnostic device may include a second inflatable member (231), wherein the second inflatable member is connected to the first inflatable member and/or the pressure source. A second conduit (233) may be connected to the second inflatable member adapted to inflate the second inflatable member to a diameter greater than a diameter of the first inflatable member.

The first inflatable member and the second inflatable member may have a deflated state (230a), in which air pressure inside the inflatable member(s) is relatively low and an inflated state (230b) in which air pressure inside the inflatable member(s) is relatively higher and in which the inflatable member(s) has become expanded/distended.

In some embodiments, the second inflatable member is an annular member surrounding a portion of the first inflatable member at the distal end of the annular body. In some embodiments, the second inflatable member may be located at the distal end of the first inflatable member. This may help, for example, when trying to obtain images of the interior of the bodily orifice of the subject, for example, the cervix. The second inflatable member may be smaller than the first inflatable member.

In some embodiments of the invention, the medical diagnostic device may include an inflatable anchoring member operable to anchor the annular body in the bodily orifice, thereby holding a position of the annular body for a period of time. For example, the inflatable anchoring member may comprise an additional inflatable member(s) (e.g., as described with respect to FIGS. 3-4D). The inflatable anchoring member may be configured to apply pressure to a small portion of the interior of the bodily orifice. The inflatable anchoring member may allow the position of the medical diagnostic device to be stabilized. The inflatable anchoring member, when inflated, may prevent the medical diagnostic device from unintentionally moving out of, or moving within, the bodily orifice of the subject. The inflatable anchoring member may therefore enhance image capture of the first imaging device (by reducing unnecessary movement of the first imaging device). The inflatable anchoring member may additionally support image capture by separating the bodily orifice in the vicinity of the first imaging device, and, for the same reason, may further aid in acquiring swabs from the cervix or other body parts. The inflatable anchoring member may be particularly useful in cases of vaginal wall prolapse or high vaginal secretion. The inflatable anchoring member may not be required for the full length of an examination using the medical diagnostic device (or may not be required at all).

According to some embodiments of the invention, the medical diagnostic device may include legs or wings configured to anchor the annular body. For example, the inflatable anchoring member may comprise legs or wings. The legs or wings may, when expanded or extended, provide improved access to and visualization of the bodily orifice of the subject (which may be particularly useful in cases where the subject is obese or suffers from a prolapse).

Upon insertion of annular body, and first inflatable member connected thereto, into the bodily orifice of a subject, the first inflatable member may be inflated to push walls of the bodily orifice away from each other to provide a wider field of view for first imaging device as compared to a field of view that may be provided by an annular body of a device that has no first inflatable member.

In some embodiments, upon insertion of annular body and first inflatable member and second inflatable member connected thereto into the bodily orifice of a subject, first inflatable member and second inflatable member may be inflated to push walls of the bodily orifice away from each other to provide a wider field of view for first imaging device as compared to a field of view that may be provided by an annular body of a device that has only a first inflatable member.

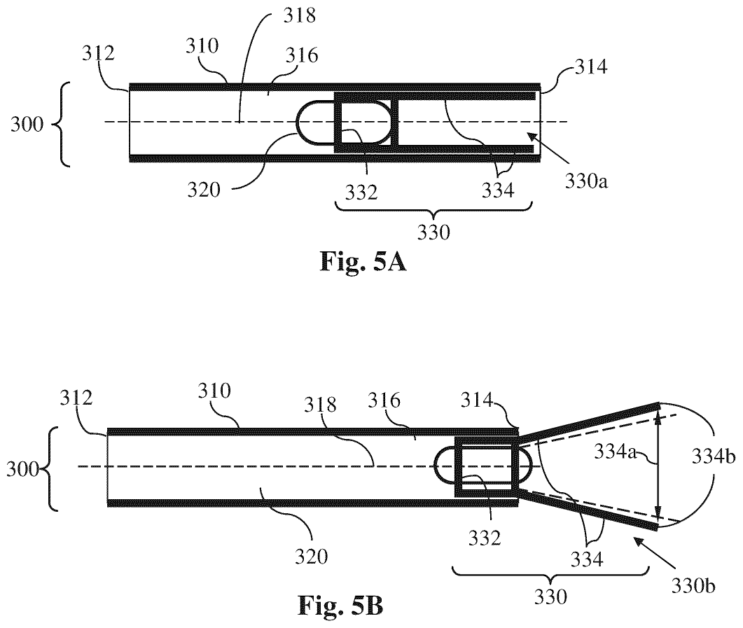

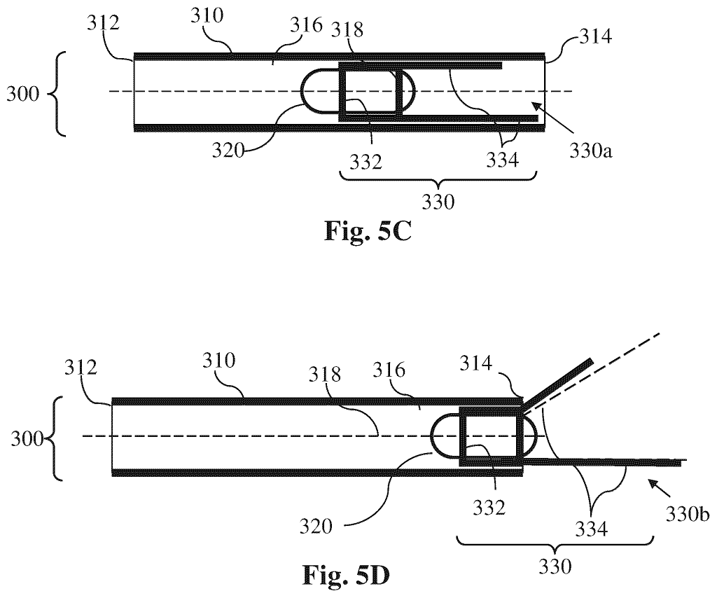

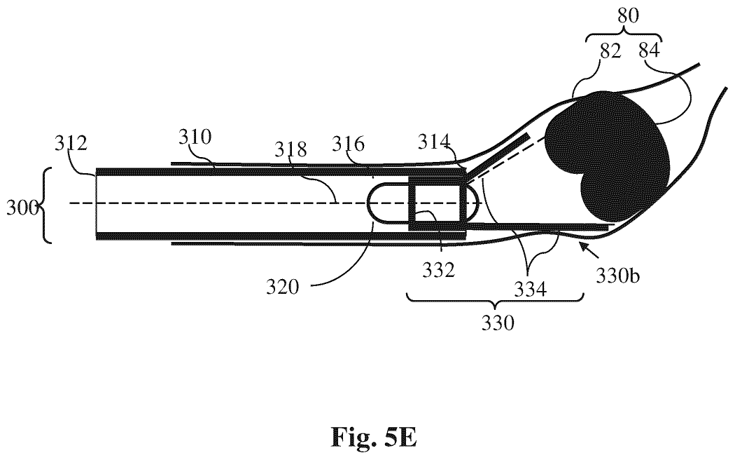

Reference is now made to FIGS. 5A-5E, which are schematic illustrations of a medical diagnostic device (300) including a resilient or flexible (non-inflatable) member (330), according to some embodiments of the invention.

According to some embodiments of the invention, a resilient member may be an embodiment of an expandable member, which may be configured to fit tightly within a hollow interior (316) of an annular body (310). The annular body may have a proximal end (312), a distal end (314), a hollow interior (316) and a central longitudinal axis (318). In some embodiments, the resilient member may include a support (332) (e.g., annular support) fitting tightly within the interior of the annular body, and a plurality of opposing longitudinal elements (334) each connected at one end to a support. The longitudinal elements may be configured to have

The resilient member may be movable with respect to annular body between its non-protruded state (330a) and its (partly) protruded state (330b). The support of the resilient member may be pushed, for example by a first imaging device (320), towards a distal end (314) of the annular body to cause longitudinal elements of resilient member to protrude from the distal end of annular body.

In some embodiments, in an initial non-protruded state, the resilient member may be fully accommodated within an annular body. Resilient member may be preloaded such that when longitudinal elements of resilient member protrude from distal end of annular body, at least one of longitudinal elements recoils away from central longitudinal axis of annular body such that a distance (334a) formed between free ends of longitudinal elements is greater than an outer diameter of distal end of annular body.

For example, the resilient member may be preloaded such that both longitudinal elements recoil away from central longitudinal axis of annular body. In another example, the resilient member may be preloaded such that only one longitudinal element may recoil away from central longitudinal axis of annular body.

In various embodiments, longitudinal elements of the resilient member may have the same or different lengths. In some embodiments, longitudinal element(s) recoiled from central longitudinal axis of annular body may push walls of the bodily orifice away from each other to provide a wider field of view for first imaging device as compared to a field of view that would be provided by an annular body of a device that has no resilient member.

The imaging device may be used to observe the bodily orifice of the subject (80), which may, for example, include the walls of the bodily orifice (e.g., vaginal walls) (82), and/or the cervix (84).

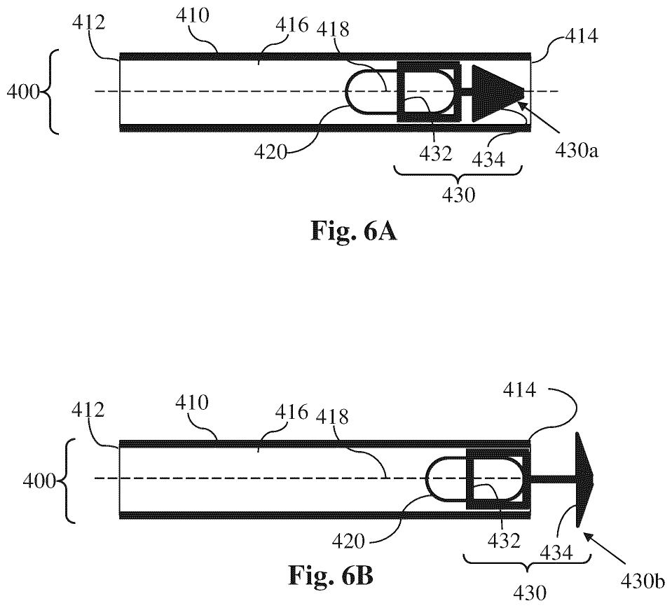

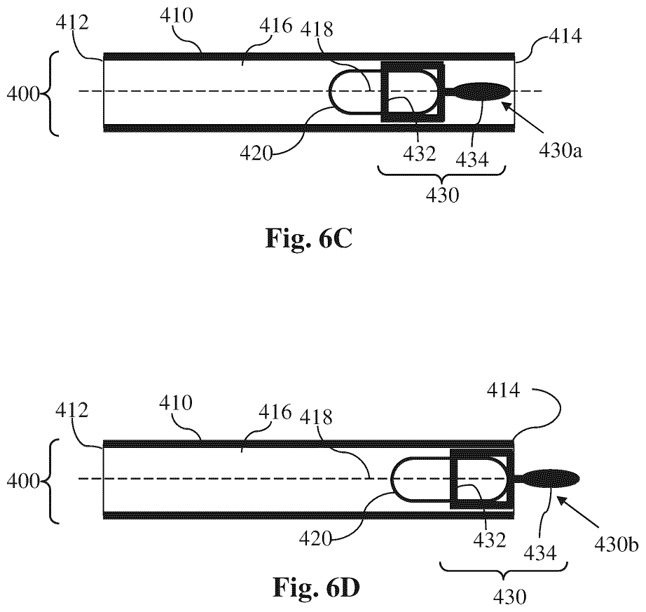

Reference is now made to FIGS. 6A-6D, which are schematic illustrations of a medical diagnostic device (400) including a swabbing member (430), according to some embodiments of the invention.

According to some embodiments of the invention, medical diagnostic device may include a swabbing member, being fully accommodated within an interior (416) of an annular body (410) and configured to protrude from and withdraw into a distal end (414) of the annular body. The annular body may have a proximal end (412), a distal end (414), a hollow interior (416) and a central longitudinal axis (418). An imaging device (420) may be disposed within the annular body.

The swabbing member may fit tightly within an interior of the annular body. In some embodiments, the swabbing member may include a support (432) fitting tightly within interior of annular body, and a swab (434) connected to the support.

The swabbing member may be movable with respect to annular body between its non-protruded state (430a) and its (partly) protruded state (430b). The support of the swabbing member may be pushed, for example by a first imaging device (420), towards the distal end of the annular body to cause the swab of the swabbing member to protrude from the distal end of the annular body.

The swabbing member may be operable from the base to push the support of the swabbing member to cause the swab of the swabbing member to protrude from the distal end of the annular body.

The swab of the swabbing member may be configured to change shape when protruded, compared to when not protruded. For example, the swab may expand when protruded, as in FIG. 6B. This may increase the efficacy of the swab at obtaining samples.

The swabbing member may include different types of swabs. For example, the swab may include a Papanicolaou (PAP) test swab. In another example, the swab may include a swab for uterine and vaginal swabbing. In another example, the swab may include a swab for vaginal and/or uterus cervix cleaning and/or collection of vaginal and/or uterus cervix excretions.

In embodiments, a dual PAP/human papilloma virus (HPV) swab configuration may be located at the distal end of the annular body. The PAP+HPV configurations may enable the camera/imaging device to view/capture images, while performing the PAP sampling. The PAP swab may have two or more wings with an angle smaller than 180 degrees between the wings.

Reference is now made to FIGS. 7A-7F, which are schematic illustrations of a medical diagnostic device (500) including a longitudinal channel (534), according to some embodiments of the invention. The device may include a longitudinal channel within an annular body (510) or adjacent to an annular body. The annular body may have a proximal end (512), a distal end (514), a hollow interior (516) and a central longitudinal axis (518). An imaging device (520) may be disposed within or insertable into the annular body.

The longitudinal channel may extend from a proximal end (512) to a distal end (514) of the annular body. Longitudinal channel may be configured to receive a tool (70) and to enable insertion of the tool into a bodily orifice of a subject. The tool may include, for example, a test swab, biopsy forceps, etc. In some embodiments, the longitudinal channel may be configured to receive a tool within an envelope (60) and to enable insertion of the tool and the envelope into the bodily orifice.

In some embodiments, a separate work channel may be detached and reattached from the annular body by a user. The separate work channel may be constructed of flexible material.

In some embodiments, the longitudinal channel may include an arc-shaped longitudinal channel surface (532) connected to an inner longitudinal surface (511) of the annular body such that a hollow channel interior is formed between longitudinal channel surface and inner longitudinal surface of annular body. Channel interior may receive the tool and/or the tool within the envelope. In some embodiments, the longitudinal channel surface may be an integral part of a longitudinal surface of annular body.

In some embodiments, the longitudinal channel may include an arc-shaped longitudinal channel surface connected to an outer longitudinal surface of annular body such that a hollow channel interior is formed between longitudinal channel surface and outer longitudinal surface of annular body. The channel interior may receive the tool and/or the tool within the envelope.

In some embodiments, the longitudinal channel may be formed in a wall of the annular body.

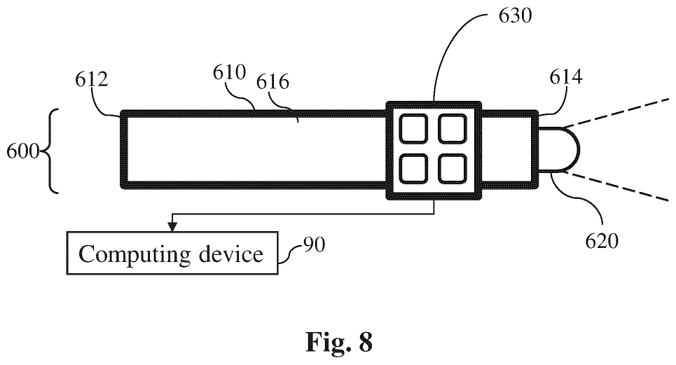

Reference is now made to FIG. 8, which is a schematic illustration of a medical diagnostic device (600) including one or more sensors (630), according to some embodiments of the invention.

One or more sensors may be disposed on an external longitudinal surface of an annular body (610). The annular body may have a proximal end (612), a distal end (614), a hollow interior (616) and a central longitudinal axis. An imaging device (620) may be disposed within or insertable into the annular body.

In some embodiments, one or more sensors may be configured to measure various parameters of the internal environment of the bodily orifice. For example, one or more sensors may be configured to measure impedance, pH, pressure in an intravaginal or rectal cavity, etc.

In some embodiments, one or more sensors may be connectable to a remote computing device (90) (e.g., as described in FIG. 1) and may transmit signals from the sensor(s) to remote computing device. The remote computing device may, for example, analyze the measured data and/or provide feedback to the user.

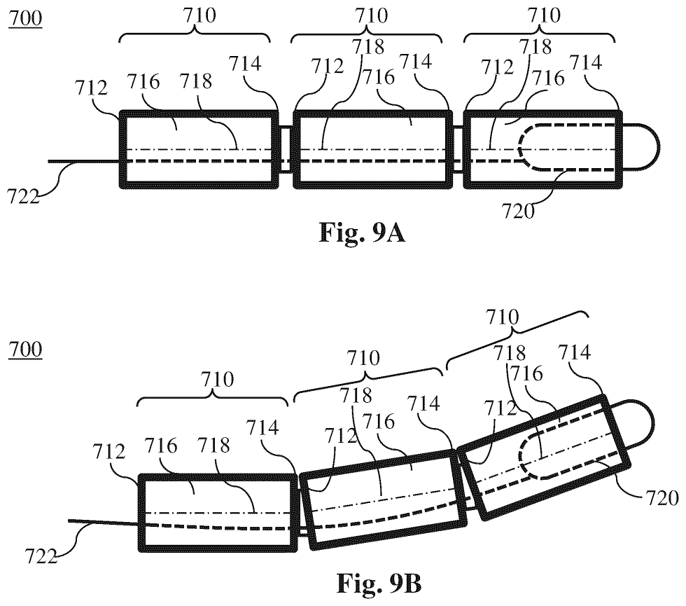

Reference is now made to FIGS. 9A and 9B, which are schematic illustrations of a

medical diagnostic device (700), wherein the annular body includes two or more interconnected annular segments (710), which may permit the annular body to be flexed according to some embodiments of the invention.

According to some embodiments of the invention, the medical diagnostic device may include an annular body comprising two or more interconnected annular segments. Each of the annular segments may include a proximal end (712), a distal end (714), a hollow interior (716) and a central longitudinal axis (718). Each pair of adjacent annular segments may include a first adjacent annular segment and a second adjacent annular segment, wherein the second adjacent annular second segment may be rotatably connected at its proximal end to distal end of the first adjacent annular segment of the respective pair such that central longitudinal axis of the second adjacent annular segment may be displaced with respect to central longitudinal axis of the first adjacent annular segment of the respective pair.

An imaging device (720) may be insertable into interiors of annular segments. The imaging device may be movable within interiors of annular segments. In some embodiments, the medical diagnostic device may include a guiding wire (722) connected to the first imaging device and configured to guide the movement of first imaging device through interiors of annular segments.

According to some embodiments of the invention, the medical diagnostic device may include a DC power supply comprising at least one of a battery, electric bus power supply or USB interface and induction power supply interface, a pump connected to the first conduit and to the second conduit, and sensors configured to control operation of the pump. In some embodiments, the DC power supply, pump and the sensors may be disposed in hollow interior of a basc.

In some embodiments, one or more of the annular segments may additionally include an expandable member according to embodiments of the invention. For example, the most distal annular segment may include an expandable member, such as an inflating member or a resilient member.

In an embodiment depicted in FIG. 10, a pump (1010) may be configured to regulate the flow of pressurized gas through a first conduit (1020) to inflate and deflate a first inflatable member (1030), mounted on an annular body (1040), and/or regulate the flow of pressurized gas through a second conduit to inflate and deflate a second inflatable member. Regulation of air flow may be further regulated through an air release valve (1050), to which the first conduit (and/or the second conduit) may be attached or pass through.

According to some embodiments of the invention, a distal end (1014) of annular body may incorporate a clear or translucent cover over a first imaging device. A base may be provided with a clear or transparent cover over a second imaging device.

FIG. 10 shows a depiction with the inflatable member in a deflated state and a further depiction with the inflatable member in an inflated state. The embodiment of FIG. 10 may be fully or partially housed within a base, e.g., the pump, conduit, and possibly part of the annular body may be housed within a hollow interior of the base, e.g., the bases of FIGS. 2A-2C.

According to some embodiments of the invention, the first imaging device may further comprise an integrated light source, such as a light emitting diode (LED) to illuminate the subject, e.g., to illuminate a cervix. In embodiments, different wavelengths of light may be selected, such as by using LEDs with different wavelengths and providing hand-held or remote controls for selecting and deselecting the LEDs.

According to some embodiments of the invention, the first imaging device may be adapted to be rotatable in at least three planes of motion. The first imaging device may provide a field of view ranging from 5 mm to 30 cm.

According to some embodiments of the invention, the first imaging device may be adapted to scan and obtain images of the internal environment of the bodily orifice of the subject in at least three planes of motion according to a predetermined pattern.

According to some embodiments of the invention, the first imaging device may be adapted to obtain images of a predefined set of anatomical features. For example, the first imaging device may capture a panoramic pelvic view, a vulvar-vestibular panoramic view, a vagina panoramic view, a posterior fornix view, an anterior fornix view, etc.

According to some embodiments of the invention, medical diagnostic device may include a fiber-optic cable insertable into the hollow interior of the annular body, wherein the fiber-optic cable comprises a proximal end and a distal end. The proximal end of the fiber-optic cable may terminate within the hollow interior of the base and the distal end of the fiber-optic cable may terminate at the distal end of the annular body. In some embodiments, the distal end of the fiber-optic cable is adapted to connect to at least one of an ultrasound probe or an imaging device (e.g., the first imaging device, or a third imaging device).

According to some embodiments of the invention, the medical diagnostic device may include a processor, and a communication unit comprising at least one of a wireless (e.g., WiFi or Bluetooth) and a non-wireless communication interface (e.g., USB). In some embodiments, the processor and communication unit are insertable in the base. In some embodiments, the processor may be a remote computing device.

The processor may be configured to control a process of the first imaging device obtaining images of the predefined set of anatomical features, generate status data relating to the process of the first imaging device obtaining images of the predefined set of anatomical features, generate and update a personalized profile of the subject from the images, and communicate the status data and the personalized profile to a remote computing device using the communication unit.

The status data may include at least an indication of successfully obtaining images of one anatomical feature from the predefined set of anatomical features and an indication of successfully obtaining images of all anatomical features from the predefined set of anatomical features.

According to some embodiments of the invention, medical diagnostic device may include at least one of an accelerometer and a gyroscope to measure orientation and location of the first imaging device within the bodily orifice of the subject.

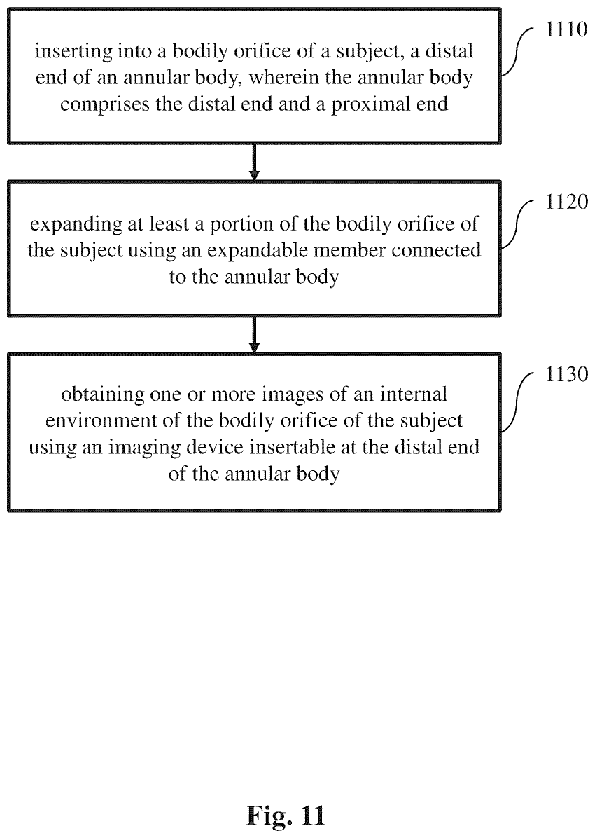

FIG. 11 shows a flowchart representing a method for screening or imaging of an orifice of a subject according to embodiments of the present invention, and which may use devices as explained in this invention.

Operation 1110 may involve inserting into a bodily orifice of a subject, a distal end of an annular body, wherein the annular body comprises the distal end and a proximal end. The annular body may be an annular body of a medical diagnostic device according to any embodiment of the present invention. The bodily orifice may be any suitable orifice, for example, the vagina of a subject.

Operation 1120 may involve expanding at least a portion of the bodily orifice of the subject using an expandable member connected to the annular body. The expandable member may be an inflatable member or a resilient member according to embodiments of medical diagnostic devices of the present invention.

Operation 1130 may involve obtaining one or more images of an internal environment of the bodily orifice of the subject using an imaging device insertable at the distal end of the annular body.

In some embodiments, expanding at least a portion of the bodily orifice of the subject using an expandable member involves inflating an inflatable member via a conduit connected to the inflatable member, wherein the inflatable member is an annular member which surrounds the annular body.

In some embodiments, the method further involves inflating a second inflatable member, connected to the inflatable member, e.g., via a second conduit connected to the second inflatable member, wherein the second inflatable member is an annular member configured to be inflated to diameter greater than a diameter of the inflatable member.

In some embodiments, the method further involves inflating an inflatable anchoring member to anchor the annular body in the bodily orifice.

In some embodiments, expanding at least a portion of the bodily orifice of the subject using an expandable member comprises extending a resilient member wherein the resilient member comprises: a support, configured to fit within the interior of the annular body; and at least two longitudinal elements, each connected at one end to the support; wherein the resilient member is preloaded such that when the longitudinal elements protrude from the distal end of the annular body, at least one of the longitudinal elements recoils away from a central longitudinal axis of the annular body such that a distance formed between frec ends of the longitudinal elements is greater than an outer diameter of the distal end of the annular body.

In some embodiments, the method further involves swabbing, with a swab of a swabbing member, an area of the bodily orifice of the subject, the swabbing member comprising: a support, configured to fit within the interior of the annular body; and the swab connected to the support; wherein the swab is configured to protrude from and withdraw into the distal end of the annular body.

In some embodiments, the method further involves inserting a tool or a tool with an envelope through a longitudinal channel, the longitudinal channel being within the annular body and extending between the proximal end and the distal end of the annular body.

In some embodiments, the method further involves measuring one or more parameters indicative of the internal environment of the bodily orifice of the subject, using one or more sensors disposed on an exterior surface of the annular body, the one or more parameters being, for example, impedance, pH, and pressure.

In some embodiments, the annular body comprises two or more interconnected annular segments, cach having a proximal end, a distal end, a hollow interior and a central longitudinal axis; wherein in each pair of a first adjacent annular segment and a second adjacent annular segment, the second adjacent annular segment is rotatably connected at its proximal end to the distal end of the first adjacent annular segment of the respective pair such that the central longitudinal axis of the second adjacent annular segment is displaceable with respect to the central longitudinal axis of the first adjacent annular segment of the respective pair; and wherein the first imaging device may be insertable into interiors of annular segments.

In some embodiments, the method further involves obtaining one or more images of an external environment of the bodily orifice of the subject, using a second imaging device insertable into the hollow interior of a base of the medical diagnostic device.

In some embodiments, the medical diagnostic device further comprises a basc connected to the proximal end of the annular body adapted to be held by hand of the subject and comprising a hollow interior.

Medical diagnostic devices according to various embodiments of the invention

described above be used for: obtaining images of an intravaginal environment of a subject (e.g., using an imaging probe), measuring various parameters of the intravaginal environment of the subject (e.g., using one or more sensors), cleaning the intravaginal environment of the subject (e.g., using various swabs), obtaining biological samples from the intravaginal environment of the subject (e.g., using various insertion tools) and/or introducing liquids into the intravaginal environment of the subject. The disclosed device may be used by the subject, for example at a subject's home and without the need to attend a healthcare professional appointment. The images and/or the measured data obtained by the disclosed device may be analyzed by a computing device interfaceable with the disclosed devices (e.g., subject's table, smartphone, personal computer) and/or transmitted to a healthcare professional for analysis. The disclosed device may be used by a healthcare professional at a healthcare facility while eliminating a need for dedicated colposcopy equipment.

In some embodiments, a method of gynecological examination or self-examination uses

image analysis to recognize anatomical landmarks that may be used to implement a computer-assisted protocol for gynecological examination or self-examination. Onboard or remote processors may be adapted to assist positioning a device according to the invention to provide an optical range of data to recognize different anatomical land marks of the pelvis, including a panoramic pelvic view, vulvar-vestibular-panoramic view, vagina-panoramic view, (with the distal end of annular body including imaging device fully inserted between the labia minor lips); posterior fornix view (with annular body inserted along the length of vaginal tube; and anterior fornix view (with direct view of the cervix (distal head of the head retracted and advanced to the pubis bone direction). The one or more onboard or remote processors may analyze an image obtained by the first imaging device to determine whether the image or view corresponds to a predetermined standard view and provide an indication to the user (which may be the subject herself) that a specific stage of the examination is complete.

Systems and methods of the present invention may improve existing medical visualization technology, for example colposcopy technology. For example: self-operation and self-adjustment by a subject may be allowed/enabled; only part of the bodily orifice of the subject need be expanded to obtain images, e.g., only part of the vaginal canal need be expanded; image capture of the interior of the bodily orifice may be improved, e.g., image capture of the cervix of a subject may be improved; comfort of the subject may be improved during use (discomfort may be alleviated or reduced); diagnosis of disease and cancer (e.g., cervical cancer) may be improved; images may be automatically transferred to an exterior computing device; visualization of the bodily orifice (e.g., vagina or cervix) may be particularly improved or enabled in cases of tilted uteri, lax vaginal walls, larger body size of the subject, obesity of the subject, prolapse of the bodily orifice, or high vaginal secretion; examination may be remote (e.g., the physician may be in a different location to the patient/subject during examination); the medical diagnostic device of the present invention may be sent to a patient/subject directly (e.g., by post); the need for speculums may be removed (which may, for example, improve the comfort, dignity, and/or health outcomes of a patient); and it may be possible to provide care with greater urgency (e.g., remote examination may mean a subject/patient does not need to travel as far for examination).

It is noted that although various features of a medical diagnostic device according to various embodiments of the present invention are described above in the context of a single embodiment, these features may be also provided separately or in any suitable combination.

In the above description, an embodiment is an example or implementation of the invention. The various appearances of “one embodiment”, “an embodiment”, “certain embodiments” or “some embodiments” do not necessarily all refer to the same embodiments.

Although various features of the invention can be described in the context of a single embodiment, the features can also be provided separately or in any suitable combination. Conversely, although the invention can be described herein in the context of separate embodiments for clarity, the invention can also be implemented in a single embodiment. Certain embodiments of the invention can include features from different embodiments disclosed above, and certain embodiments can incorporate elements from other embodiments disclosed above. The disclosure of elements of the invention in the context of a specific embodiment is not to be taken as limiting their use in the specific embodiment alone. Furthermore, it is to be understood that the invention can be carried out or practiced in various ways and that the invention can be implemented in certain embodiments other than the ones outlined in the description above.

The invention is not limited to those diagrams or to the corresponding descriptions. For example, flow need not move through each illustrated box or state, or in exactly the same order as illustrated and described. Meanings of technical and scientific terms used herein are to be commonly understood as by one of ordinary skill in the art to which the invention belongs, unless otherwise defined. While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

1. A medical diagnostic device, comprising:
an annular body having a proximal end, a distal end and a hollow interior, the distal end is configured to be inserted into a bodily orifice of a subject;
a first inflatable member connected to the annular body, the first inflatable member is configured to expand at least a portion of the bodily orifice of the subject;
a base connected to the proximal end of the annular body adapted to be held by hand of the subject and comprising a hollow interior;
a first imaging device positioned in said hollow interior of the annular body, wherein the first imaging device is configured to obtain one or more images of an internal environment of the bodily orifice of the subject;
wherein said first imaging device is configured to be movable within the hollow interior of the annular body in one or more of a longitudinal direction, tilted with respect to central longitudinal axis of annular body, rotated about the central longitudinal axis in a plane parallel to the axis and rotated about the central longitudinal axis in a plane perpendicular to the central longitudinal axis of annular body.

2. The device of claim 1, comprising controls on the base adapted to be manipulated by hand to actuate the first imaging device.

3. The device of claim 1, comprising controls on the base adapted to be manipulated by a remote controller to actuate the first imaging device.

4. The device of claim 1, further comprising:
a first conduit connected to the first inflatable member and adapted to inflate the first inflatable member;
wherein the first inflatable member is an annular member surrounding the annular body.

5. The device of claim 1, further comprising controls, operable from the base, configured to perform one or more of:
a. inflate and deflate the first inflatable member at different speeds;
b. inflate and deflate the first inflatable member according to the size and anatomy of the bodily orifice.

6-11. (canceled)

12. The device of claim 1, further comprising a motor in said base and wherein the first imaging device is connected to said motor in the base configured to move the first imaging device.

13. The device of claim 1, further comprising:
a swabbing member fitting within the interior of the annular body, being fully accommodated within the interior of the annular body and configured to protrude from and withdraw into the distal end of the annular body, the swabbing member comprising:
a support; and
a swab connected to the support;
wherein the swabbing member is operable from the base to push the support of the swabbing member to cause the swab of the swabbing member to protrude from the distal end of the annular body.

14. (canceled)

15. The device of claim 1, further comprising a longitudinal channel within the annular body and extending between the proximal end and the distal end of the annular body, wherein the longitudinal channel is configured to receive a tool or a tool within an envelope.

16-19. (canceled)

20. The device of claim 1, comprising one or more sensors disposed on an exterior surface of the annular body, the one or more sensors configured to measure a set of parameters of the internal environment of the bodily orifice of the subject comprising at least one of temperature, impedance, pH and pressure.

21. The device of claim 1, wherein:
said annular body is comprised of two or more interconnected annular segments, each having a proximal end, a distal end, a hollow interior and a central longitudinal axis;
wherein in each pair of a first adjacent annular segment and a second adjacent annular segment, the second adjacent annular segment is rotatably connected at its proximal end to the distal end of the first adjacent annular segment of the respective pair such that the central longitudinal axis of the second adjacent annular segment is displaceable with respect to the central longitudinal axis of the first adjacent annular segment of the respective pair; and
wherein the first imaging device may be insertable into interiors of annular segments.

22. (canceled)

23. The device of claim 4, comprising:
a DC power supply comprising at least one of a battery, electric bus power supply interface and induction power supply interface;
a pump connected to the first conduit and to a second conduit; and
sensors configured to control operation of the pump;
wherein the DC power supply, the pump and the sensors are insertable into the hollow interior of the base.

24-28. (canceled)

29. The device of claim 1, wherein the first imaging device is adapted to be rotatable in at least three planes of motion, and to provide a field of view ranging from 5 mm to 30 cm.

30. The device of claim 1, wherein the first imaging device is further adapted to:
scan and obtain images of the internal environment of the bodily orifice of the subject in at least three planes of motion according to a predetermined pattern.

31. The device of claim 1, wherein the first imaging device is further adapted to obtain images of a predefined set of anatomical features, comprising at least a panoramic pelvic view, a vulvar-vestibular panoramic view, a vagina panoramic view, a posterior fornix view, and an anterior fornix view.

32. The device of claim 1, further comprising:
a processor; and
a communication unit comprising at least one of a wireless and a non-wireless communication interface;
wherein the processor and communication unit are insertable in the base.

33. The device of claim 32, wherein the processor is configured to:
control a process of the first imaging device obtaining images of a predefined set of anatomical features;
generate status data relating to the process of the first imaging device obtaining images of the predefined set of anatomical features;
generate and update a personalized profile of the subject from the images; and
communicate the status data and the personalized profile to a remote computing device using the communication unit.

34. The device of claim 33, wherein the status data comprises at least an indication of successfully obtaining images of one anatomical feature from the predefined set of anatomical features and an indication of successfully obtaining images of all anatomical features from the predefined set of anatomical features.

35. The device of claim 1, further comprising at least one of an accelerometer and a gyroscope to measure orientation and location of the first imaging device within the bodily orifice of the subject.

36. The device of claim 1, wherein the first imaging device further comprises an integrated light source.

37-59. (canceled)

60. The device of claim 1, further comprising at least one of an ultrasound probe or a third imaging device.
